(19) United States
(12) Patent Application Publication
(10) Pub. No.: US 2011/0315175 A1
(43) Pub. Date: Dec. 29, 2011

(54) Overhead sanitization system

(76) Inventors: Christopher Lella, Manalapan, NJ (US); Shari Lella, Manalapan, NJ (US)

(21) Appl. No.: 13/067,270

(22) Filed: May 20, 2011

Related U.S. Application Data
(60) Provisional application No. 61/396,002, filed on May 21, 2010.

(51) Int. Cl. B08B 3/02

(52) U.S. Cl. 134/56 R

(57) ABSTRACT

An overhead sanitization system utilizing ceiling embedded control valves and nozzle heads operative to provide an antibacterial spray under the control of a central computer processing unit monitoring the fluid level within an antibacterial solution storage vessel, and with the control valves and heads being supplied with the antibacterial solution from the storage vessel under the computer unit's control of a pressurization pump coupled to the storage vessel by an included impurity eliminating filter.

### CROSS-REFERENCE TO RELATED APPLICATIONS

A Provisional Patent Application covering the invention described herein was filed on May 21, 2010, and assigned Serial No. 61/396,002.

### STATEMENT REGARDING FEDERALLY SPONSORED RESEARCH OR DEVELOPMENT

Research and development of this invention and Application have not been federally sponsored, and no rights are given under any Federal program.

### REFERENCE TO A MICROFICHE APPENDIX

Not Applicable

### BACKGROUND OF THE INVENTION

1. Field of the Invention

This invention relates to a non-sticky, non-staining, eco-friendly sanitization system, in general, and to such a system for use at a fixed or movable structural installation, in particular.

2. Description of the Related Art

As will be appreciated, more and more people every day are becoming more and more germophobic.

It is not unusual, for example, for a situation to develop where one sick child in a classroom infects the rest of the class to the extent that many others become ill. Sitting in a waiting room of a crowded medical office to wait to see the doctor can be an experience where the patient leaves in worse condition than before, simply by picking up the germs of others who may similarly be waiting their turn to be seen and treated. Passengers in airplanes, trains and buses commonly arrive at their destinations feeling more poorly than before, just as a result of absorbing germs and bacteria that may be coming off of other passengers—even where attempts of their covering up are apparent. To such an extent, as a result, it is not unusual for one to walk around any city in this or any other country, and note the ever increasing number of people wearing face masks in an attempt to protect one's self against unsanitary conditions. Subways and train stations, bus terminals and airports are nowheres unique as places where germs and bacteria may be present, as similar venues for becoming ill exist wherever large groups of people congregate—as in stadiums, auditoriums, theaters, and large closed enclosures.

Hospital rooms, hospitals, and nursing homes, for instance, recognizing the problems that can ensue, often attempt to sanitize and disinfect public areas after a workday has ended. The mopping of floors, the cleaning of bathrooms, the wiping of surfaces here and there, generally represent their attempts at sanitizing the areas in question. But those methods leave much to be desired, especially in an age where the proprietors are in an economic fix, unable to disburse for these services funds which may not be available, or which are tight in their distribution. Purchases of cleaning machines and equipments have been curtailed for similar economic reasons, with the overall result being an increase in the amount of complaints being made of bacterial and other germ infections.

One need only read a newspaper or see television news to learn of one or another instance of hundreds of passengers on cruise ships, for example, becoming infected and ill to such an extent as to be confined during their various vacation trips.

### OBJECTS OF THE PRESENT INVENTION

It is an object of the present invention, therefore, to provide an arrangement which allows for a simple and effective sanitization of all those areas where people tend to congregate, and where they are at the greatest risk of becoming sick or otherwise infected.

It is object of the invention, also, to provide this arrangement in a manner where the sanitization provided can be controlled, according to the size of the room and the number of inhabitants.

It is another object of the invention to provide this control automatically or manually, depending upon the risk of illness that might follow.

### SUMMARY OF THE INVENTION

As will become clear from the following description, the present invention describes an overhead sanitization system which can be installed in a ceiling construction in a manner analogous to a fire-extinguishing sprinkler system. But, instead of dispensing a fire-extinguishing water stream, the system of the present invention dispenses a dense, fine mist antibacterial spray. Also designed to be portable for use in buses, trains and airplanes (or other relatively small areas that are in movement), the system of the invention will be seen to include a pressurized pump able to dispense this fine mist in manner similar to that of an aerosol spray. Depending upon the location of use, and the size area where the spray is to be effective, activation can be had by way of a manually controllable switch, or to be activated in more spacious areas under computer control. As will be seen, a holding tank (such as a steel drum) can store the antibacterial spray until called upon to be dispensed by the pressurized pump. Where computer activation is had, the teachings of the invention are equally applicable to shut down the entire system operation in the event of any malfunction.

### BRIEF DESCRIPTION OF THE DRAWING

FIG. 1 represents a sanitization system, partially in block form, helpful in an understanding of the teachings of the present invention.

### DETAILED DESCRIPTION OF THE INVENTION

The overhead sanitization system—whether permanently installed in the ceilings of hospitals, hospital rooms, nursing homes, schools, medical offices, stadiums, or in such movable systems as buses, trains, and airplanes, for example—includes a storage vessel 10, a filter 12, a pressurization pump 14, a central computer processing CPU control 16, and one or more control valves 18 and nozzle heads 20. Three sets of control valves and nozzle heads are shown at “A”, as in various locations on the first floor of a school facility, three sets of control valves and nozzle heads are shown at “B” for various locations on the second floor of the school, and three sets of control valves and nozzle head are illustrated at “C” for locations at upper floors, serially, at the school, location.

In a preferred embodiment of the invention, the storage vessel 10 may be constructed of a plastic material selected to be resistant to the antibacterial spray; but where increased durability is desired, a stainless steel fabrication can be employed. Indicators of different types can be employed to reveal the amount of antibacterial solution in the storage vessel, which is intended for easy access filling through its top. An electronic sensor output coupled to the storage vessel 10 provides an input to the central computer to indicate such liquid level, as by way of the flow-line 30. When the preferred computer control is utilized, as at 16, the amount of cycles of use can be predicted and calculated, so that indications are provided as to when the storage vessel is to be refilled. An emergency shut-off control for the computer system is indicated at 32.

To prevent any impurities present in the antibacterial spray from clogging the overhead control valves, a filter 12 is employed, as an intermediary from the storage vessel 10. A flow sensor incorporated either within the filter 12 or at its output couples to the central computer control 16 to monitor the flow of the antibacterial solution to the pressurization pump 14. As will be appreciated, this allows the computer to shut down the pump in the event insufficient flow is present. Such input into the computer from the filter 12 is shown along the flow indicator line 34, with the output “ON/OFF” control to the pressurization pump 14 being shown along the line 36.

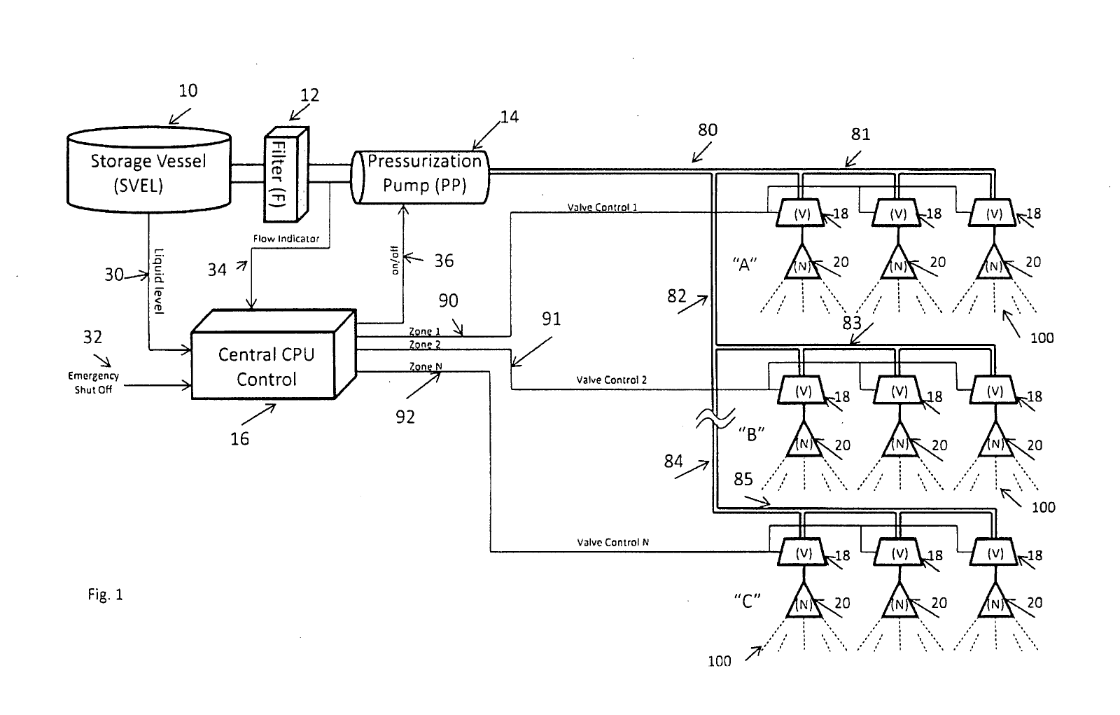

In operation, the pressurization pump 14, once controlled to its “ON” condition by the central computer control, provides the force to deliver the antibacterial solution to that location where the mist is to be created—namely, at each control valve, and on each floor where applicable, as shown by the duct conduits 80-81, 80-82-83, and 80-82-84-85. In this preferred construction, the pressurization pump 14 is not only controlled by the central computer 16 but operates with a “fail-safe” mode operative to shut down the pump in the event of a communication breakdown with the central computer. As set out in FIG. 1, each zone of the floor (i.e., each area to be sanitized) has its own set of component valves providing the antibacterial spray. Such valves'are similarly controlled by the central computer 16, able to be wired independently or in parallel with other valves. Flow, lines 90, 91 and 92 identify the computer controls for the 1st, 2nd, and Nth zone floors of the structure, each with its independent valve locations 18. As will be appreciated by those skilled in the art, the number of valves 18 that can be operated simultaneously depends upon the particular nozzle head selected for the resultant spray, and the size of the pressurization pump 14.

Because different areas of the fixed or moving structure may require greater disinfecting than others, different sized nozzle heads may be in place to control the area of spread and the volume of spray to be dispensed on any one disinfecting application. Such spread is indicated in FIG. 1 by the reference numeral 100.

As will be understood from the foregoing, full control of the disinfecting spray operation is controlled by the central computer 16. Flow velocity and solution storage levels are monitored there and governed, while the programming for the computer controls the system operation in dispensing whatever amount of solution is desired, at any one or at multiple times in accordance with the schedule desired for the particular facility in question. At the same time, which control valves are to be opened, and which are to be opened at specific time intervals can likewise be effected under programming control. Alternatively, and in a more simplified system arrangement, the computer control can simply be deactivated and operation continue as before, once each valve is set to an open condition before energizing the entire system operation.

As will thus be apparent, automatic overhead sanitization can be provided in all area where required at the end of a work day or work schedule simply, automatically, and on a regular schedule of operations to effectuate the needed control in sanitizing areas to prevent not only germs and bacteria from developing, but to eliminate any presence of odors or other smells which may characterize an area of use—such as in public restroom facilities, and especially at large stadium and arena locations. Where manual, rather than automated control is desired, the central computer control can simply be switched ON or OFF as needed.

While there have been described what are considered to be preferred embodiments of the invention, it will be readily appreciated by those skilled in the art that modifications can be made without departing from the scope of the teachings herein. For at least such reason, therefore, resort should be had to the claims appended hereto for a true understanding of the invention.

We claim:

1. An overhead sanitization system comprising:
a storage vessel containing an antibacterial solution, a filter, a pressurization pump, a central computer processing unit, and one or more control valves and nozzle heads embedded at a ceiling location;
wherein the central computer processing unit controls the storage vessel to supply a predetermined amount of antibacterial solution to the filter, wherein the filter separates out any impurities from the antibacterial solution in feeding the antibacterial solution to the pressurization pump, wherein the pressurization pump provides a force to deliver the antibacterial solution passing through the filter to preselected ones of the control valves in the ceiling location, wherein the nozzle heads are coupled with the control valves to provide an antibacterial solution spray down from the ceiling location, and wherein the computer processing unit also controls which ones of the control valves are to be operative at any given instant of time.

2. The system of claim 1 wherein each of the control valves and nozzle heads are embedded at a ceiling location in one of a fixed or movable structural installation.

3. The system of claim 2 wherein the storage vessel is constructed of one of a plastic or stainless steel fabrication.

4. The system of claim 3 wherein the central computer processing unit additionally monitors the fluid level of antibacterial solution within the storage vessel for a refilling thereof.

5. The system of claim 4, including a first electric sensor coupled between the storage vessel and the central computer processing unit in monitoring the antibacterial solution fluid level within the storage vessel.

6. The system of claim 5, also including an emergency shut-off control for the central computer processing unit.

7. The system of claim 6, additionally including a second electric sensor coupled between the filter and the central computer processing unit in monitoring the antibacterial solution fluid flow level through the filter to the pressurization pump.

8. The system of claim 7 wherein said central computer processing unit additionally controls the pressurization pump to shut down in the event the monitored antibacterial solution fluid level flow falls below a preselected amount.

9. The system of claim 1 wherein the individual nozzle heads are selected of the same or different size to provide the antibacterial solution spray characteristic desired from that nozzle.

10. The system of claim 9 wherein the control valves and nozzle heads are embedded at ceiling locations on the same or different floors of a fixed structural installation.
